(19) United States
(12) Patent Application Publication
(10) Pub. No.: US 2009/0264460 A1
(43) Pub. Date: Oct. 22, 2009

(54) CLOPIDOGREL PHARMACEUTICAL FORMULATIONS

(76) Inventors: Mamta Mishra, Hyderabad (IN); Dinesh Chakole, Nagpur (IN); Venkata Sivareddy Pallempalli, Hyderabad (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 12/426,548

(22) Filed: Apr. 20, 2009

Related U.S. Application Data
(60) Provisional application No. 61/081,542, filed on Jul. 17, 2008.

(30) Foreign Application Priority Data
Apr. 21, 2008 (IN) 983/CHE/2008

(51) Int. Cl. A61K 31/38

(52) U.S. Cl. 514/301

(57) ABSTRACT

Formulations of clopidogrel or pharmaceutically acceptable salts thereof, processes of preparation of such formulations, and methods of using them.

The present invention relates to pharmaceutical formulations comprising clopidogrel, including pharmaceutically acceptable salts thereof. The present invention further relates to formulations comprising clopidogrel, including pharmaceutically acceptable salts thereof, which provide desired bioavailability. The present invention also provides processes of preparation of such formulations, and methods of using them.

The drug compound having the adopted name “clopidogrel” has chemical names S-(+)-methyl-(2-chlorophenyl)-(6,7-dihydro-4H-thieno[3,2-C]pyrid-5-yl) acetate, or (+)-(S)-α-(2-chlorophenyl)-6,7-dihydrothieno[3,2-c]pyridine-5(4H)-acetate, and has structural Formula I.

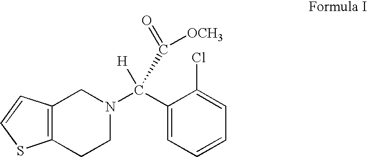

Clopidogrel is a well-known antithrombotic agent, commercially available in the products sold by Bristol-Meyers Squibb as PLAVIX™ tablets that contain 97.875 mg of clopidogrel hydrogen sulfate (the molar equivalent of 75 mg clopidogrel), 195.75 mg of clopidogrel hydrogen sulfate (the molar equivalent of 150 mg clopidogrel), and 391.5 mg of clopidogrel bisulfate (the molar equivalent of 300 mg clopidogrel).

Clopidogrel is an inhibitor of platelet aggregation, shown to decrease morbidity in people with established cardiovascular atherosclerotic disease as evidenced by stroke or transient ischemic attacks, myocardial infarction, unstable angina, or the need for vascular bypass or angioplasty.

Clopidogrel and its preparation are described in U.S. Pat. Nos. 4,529,596 and 4,847,265. U.S. Pat. No. 5,576,328 discloses methods of using clopidogrel and a pharmaceutically acceptable acid addition salt thereof, together with a pharmaceutically acceptable carrier.

International Application Publication No. WO 99/65915 discloses two crystalline polymorphic forms of clopidogrel hydrogen sulfate, referred to as Forms I and II.

U.S. Pat. Nos. 6,429,210 and 6,504,030 disclose crystalline polymorphic Form II of clopidogrel hydrogen sulfate, and processes for its preparation.

U.S. Pat. No. 6,914,141 discloses pharmaceutical tablets comprising clopidogrel bisulfate and a lubricant selected from zinc stearate, stearic acid, and sodium stearyl fumarate. U.S. Pat. Nos. 6,767,913 and 7,074,928 disclose pharmaceutical formulations comprising different polymorphic forms of clopidogrel hydrogen sulfate, and pharmaceutically acceptable excipients.

International Application Publication No. WO 2006/044548 discloses solid premixes comprising clopidogrel base, adsorbed onto at least one pharmaceutically acceptable excipient. International Application Publication No. WO 2004/098593 discloses formulations comprising amorphous clopidogrel hydrogen sulfate, either or both of calcium stearate and magnesium stearate, a non-hygroscopic additive, and at least one excipient.

International Application Publication No. WO 2007/008045 discloses formulations containing clopidogrel bisulfate and pregelatinized starch.

Several challenges exist for formulating clopidogrel or its salts into suitable dosage forms. To mention a few, clopidogrel base occurs as an amorphous semisolid paste-like mass, and because of this physical state it creates processing problems for preparing a pharmaceutical dosage form. Similarly, clopidogrel salts, particularly clopidogrel bisulfate, is extremely hygroscopic and causes picking, sticking, adhesion and other kinds of surface irregularities, leading to finished products with poor quality and appearance.

Furthermore, certain excipients enhance the inherent stickiness of the clopidogrel salts. The use of an appropriate lubricant or dry granulation (compaction) should solve the problem. However, the choice of excipients for manufacturing of clopidogrel tablets can also be important, because clopidogrel exhibits rapid degradation when co-processed with certain excipients. Hence, selection of the pharmaceutically acceptable excipients can be important. Moisture content of the formulation can be important for its polymorphic stability.

There exists a need for developing stable and bioequivalent formulations comprising clopidogrel or pharmaceutically acceptable salts thereof, wherein the active agent retains its polymorphic form and wherein the formulations are devoid of any substantial impurities upon storage. Thus there remains a need for stable pharmaceutical formulations comprising clopidogrel or a pharmaceutically acceptable salt thereof.

### SUMMARY

The present invention provides formulations of clopidogrel, including pharmaceutically acceptable salts thereof. The present invention also provides process of preparation of such formulations, and methods of using them.

In an aspect, the invention includes e pharmaceutical formulations comprising clopidogrel or a pharmaceutically acceptable salt thereof as active agent, a lubricant in an amount of about 0.5% to about 5% by weight of the formulation, comprising at least one of stearic acid, magnesium stearate, zinc stearate, calcium stearate, sodium stearyl fumarate, glycery behenate and hydrogenated castor oil, and one or more other pharmaceutically acceptable excipients, wherein the active agent is present in an amount of about 30-450 mg in the formulation.

In an aspect, the invention includes pharmaceutical formulations comprising clopidogrel or a pharmaceutically acceptable salt thereof, and sodium stearyl fumarate or hydrogenated castor oil as a lubricant.

In an aspect, the invention includes pharmaceutical formulations comprising: a) a solid premix comprising clopidogrel base, adsorbed onto at least one pharmaceutically acceptable excipient; and b) sodium stearyl fumarate as a lubricant.

In an aspect, the invention includes pharmaceutical formulations comprising clopidogrel or a pharmaceutically acceptable salt thereof and sodium stearyl fumarate as a lubricant, wherein the lubricant is present in a concentration of about 0.05 to about 8%, or about 0.5 to about 5%, by weight of the formulation.

In an aspect, the invention includes pharmaceutical formulations comprising: a) a solid premix comprising clopidogrel base, adsorbed onto at least one pharmaceutically acceptable excipient; and b) sodium stearyl fumarate as a lubricant, wherein the lubricant is present in a concentration of about 0.5 to about 5% by weight of the formulation.

In an aspect, the invention provides pharmaceutical formulations comprising at least one diluent comprising microcrystalline cellulose, at least one disintegrant comprising croscarmellose sodium, sodium starch glycolate or crospovidone, and one or more other pharmaceutically acceptable excipients.

In an aspect, the invention comprises dosage forms containing clopidogrel bisulphate and excipients, and having a water content of about 0.5 to 10%, or about 2 to 5%, or about 3%, by weight as determined by the Karl Fischer method.

In embodiments, the invention includes methods of preparing the pharmaceutical formulations of the present invention comprising clopidogrel or pharmaceutically acceptable salts thereof as the active agent.

In further embodiments, the invention provides methods of treating patients suffering from atherothrombotic events (e.g., myocardial infarction, stroke and vascular death) or acute coronary syndrome, using the pharmaceutical formulations of the present invention.

### DETAILED DESCRIPTION

The present invention relates to formulations of clopidogrel or pharmaceutically acceptable salts thereof. The present invention further relates to formulations comprising clopidogrel or pharmaceutically acceptable salts thereof which exhibit appreciable bioavailability in order to achieve the desired pharmacological action. In an aspect, formulations of the present invention are bioequivalent to a PLAVIX product. In an aspect, the present invention provides stable formulations comprising clopidogrel base. In another aspect, the present invention provides stable formulations comprising clopidogrel bisulphate.

The present invention, as discussed in various embodiments and aspects, relates to formulations comprising clopidogrel or pharmaceutically acceptable salts thereof, wherein the formulations are not only stable during the manufacturing but they also possess appreciable stability for a commercially relevant time during storage.

The term “clopidogrel” as used herein includes the compound clopidogrel, pharmaceutically acceptable salts, esters, prodrugs thereof, the active metabolites of clopidogrel and the prodrugs thereof, and their, solvates and hydrates in any polymorphic forms. The term “clopidogrel” also includes a clopidogrel base premix with one or more excipients.

The terms “salt” or “pharmaceutically acceptable salt” as used herein refer to salts of clopidogrel which are non-toxic and are commonly used in pharmaceutical practice. Such pharmaceutically acceptable salts include metal salts, salts with organic bases, salts with basic amino acids, etc. Acid addition salts such as bisulphate or hydrochloride salts and the like are also included.

The term “formulation” as used herein refers to solid dosage forms comprising clopidogrel, including pharmaceutically acceptable salts thereof, such as in the form of tablets or capsules. A formulation according to the present invention also encompasses multiparticulate formulations such as pellets, granules, minitablets, powders, and the like. Such multiparticulate formulations can be compressed or compacted into tablets, filled into capsules, or used directly.

The term “stable” as used herein refers to physical stability and chemical stability, wherein physical stability refers to retaining an original polymorphic form in the formulation and chemical stability refers to resistance to impurity generation.

The term “bioequivalent” as used herein refers to the rate and the extent of absorption of the clopidogrel or pharmaceutically acceptable salts thereof being between 80% and 125% of that obtained by administering a PLAVIX tablet of the same strength.

The term “substantially free” as used herein refers to clopidogrel bisulphate Form I containing less than about 20%, or less than about 10%, or less than about 5%, by weight of clopidogrel bisulphate Form II.

In the present invention, clopidogrel and its salts, or any other pharmaceutical form of clopidogrel, can be in a crystalline form, amorphous form, or combinations thereof. The active agent, i.e., clopidogrel or its salt can be in the form of a ‘hydrate’ or in any polymorphic forms such as amorphous, or Form I (as disclosed in WO 99/65915) or Form II (as disclosed in WO 99/65915 and U.S. Pat. Nos. 6,429,210 and 6,504,030), and any mixtures thereof.

As used herein the term “premix” refers to solid compositions, generally powders or granules, comprising clopidogrel, adsorbed onto at least one pharmaceutically acceptable excipient that is compatible with clopidogrel base.

Clopidogrel base occurs as an amorphous semisolid paste-like mass. Hence, the use of clopidogrel as an active pharmaceutical ingredient in pharmaceutical dosage forms requires that the clopidogrel base be processed so as to render it suitable for further processing to develop a pharmaceutical dosage form.

Conversion of clopidogrel base into a free-flowing solid with predefined characteristics suitable for its direct use as a medicament, or which would allow its use in processing into pharmaceutical formulations, has been a major challenge faced by the pharmaceutical scientist. Clopidogrel base, when processed together with certain pharmaceutically acceptable excipients in the presence of solvents, allows the formation of a premix composition containing clopidogrel base and having enhanced stability. These premixes are isolated in the form of stable, free-flowing powders or granules, which exhibit good processing characteristics and can be easily and conveniently processed into pharmaceutical formulations (such as, for example, tablets, capsules, and the like).

Even though the clopidogrel base premixes are obtained as stable and free-flowing solids, certain process-related problems can be encountered during further processing of that premix into pharmaceutical dosage forms like tablets. These include poor flowability of the premixes when blended with other excipients, and weight variations and sticking of compressed tablet surfaces to the punches and dies during tableting, which can cause the product to have pitting and other surface irregularities. Even though these problems are not observed during preparation of many tablet formulations, stability problems like increased impurity levels at accelerated stability storage conditions sometimes occur.

During development of stable pharmaceutical formulations of clopidogrel base premixes according to the present invention it was surprisingly found that formulations comprising sodium stearyl fumarate as a lubricant are both process feasible and stable. Further it was also observed that pharmaceutical formulations comprising clopidogrel or pharmaceutically acceptable salts can be formulated with hydrogenated castor oil and other lubricants such as stearates and glyceryl behenate without any stability and/or impurity concerns during stability studies.

The premix compositions in excipient blends comprising sodium stearyl fumarate at certain concentrations are found to be process feasible, as flowability and sticking problems as well as tablet weight variations were not observed. The concentrations of sodium stearyl fumarate that were found to be desirable are about 0.05% to about 10%, or about 0.5% to about 5%, or about 0.5% to about 2%, by weight of the total formulation; below these ranges the amount of lubricant is not sufficient to overcome sticking problems, and above the ranges sufficient hardness of the tablet is not achieved.

In an aspect, the invention includes pharmaceutical formulations comprising clopidogrel or a pharmaceutically acceptable salt thereof and sodium stearyl fumarate as a lubricant.

In an aspect, the invention includes pharmaceutical formulations comprising: a) a solid premix comprising clopidogrel base, adsorbed onto at least one pharmaceutically acceptable excipient; and b) sodium stearyl fumarate as a lubricant.

In an aspect, the invention includes pharmaceutical formulations comprising clopidogrel or a pharmaceutically acceptable salt thereof and sodium stearyl fumarate as a lubricant, wherein the lubricant is present in concentrations of about 0.5% to about 2% by weight of the formulation.

In an aspect, the invention includes pharmaceutical formulations comprising: a) a solid premix comprising clopidogrel base, adsorbed onto at least one pharmaceutically acceptable excipient; and b) sodium stearyl fumarate as a lubricant; wherein said lubricant is present in concentrations of about 0.5% to about 2% by weight of the formulation.

In an embodiment, pharmaceutical formulations according to present invention have clopidogrel bisulphate Form I as the active agent. In embodiments of the invention, the formulations are substantially free of clopidogrel bisulphate Form II.

In an embodiment, the clopidogrel or its salt used to make the formulations or contained in the formulations of the present invention is amorphous, crystalline or mixtures thereof. In an aspect, the clopidogrel or its salt used as the input active agent is in a substantially amorphous form which form is substantially retained during the manufacturing of the formulation and also during storage. In another aspect, the clopidogrel or its salt used as the input active agent is in a substantially crystalline form which form is substantially retained during the manufacturing of the formulation and also during storage. In another aspect, the clopidogrel or its salt used as the input active agent is in a substantially crystalline form, which form is converted into a substantially amorphous form during the manufacturing of the formulation, and which form remains substantially amorphous during storage for a commercially relevant time.

Certain formulations according to the present invention comprise clopidogrel base or a clopidogrel salt such as bisulphate, and excipients, and have a water content of about 2% to 10%, or about 2% to 5%, or about 3%, by weight as determined by the Karl Fischer method. It has unexpectedly been found that if the water content of the formulation is controlled within the above ranges, an undesired conversion of the specific polymorphic form of clopidogrel bisulphate can be avoided, i.e., the polymorphic stability of the active agent could be maintained, in particular the conversion of clopidogrel bisulphate Form I to Form II can be substantially prevented when a crystalline form of clopidogrel bisulphate such as Form I is used.

In an embodiment, the formulations of the present invention have a LOD (loss on drying) of less than about 12% w/w of the formulation, measured at 40-250° C. using a halogen moisture balance, or a hot air oven.

In an embodiment, the formulations of the present invention are prepared using clopidogrel or its salt having a particle size distribution such that D90 is about 0.1 μm to about 1000 μ, or about 1 μto about 500 μ, and D50 is from about 0.01 μto about 500 μ. The D90 is a particle size value, for which 90% of the particles in a powder are smaller. The D50 is mean particle size, with half of the particles in a powder being smaller. There are several ways to measure particle size distributions, including laser light diffraction methods.

In an embodiment, the formulations of the present invention do not have a total impurity content more than about 5% by weight of the label clopidogrel content, when stored under accelerated stability testing conditions of 40° C. and 75% relative humidity (RH) conditions for at least 2 months, or for about 3 months, or for about 6 months.

In embodiments of the present invention, the formulations, when stored under accelerated stability testing conditions of about 40° C. and about 75% relative humidity (RH) conditions for about 6 months, have an Impurity A content not more than about 3%, or not more than about 1%, or not more than about 0.25%, by weight of the label clopidogrel content.

In embodiments of the present invention, formulations comprising clopidogrel or a pharmaceutically acceptable salt thereof may contain one or more pharmaceutically acceptable excipients. Examples of pharmaceutically acceptable excipients as used herein include any of excipients such as diluents or fillers, binders, antioxidants, disintegrants, surfactants, lubricants, glidants, and the like.

Suitable diluents or fillers include, but are not limited to, one or more of starches, lactose, mannitol (Pearlitol™ SD200), cellulose derivatives, confectioners sugar and the like. Different grades of lactose include but are not limited to lactose monohydrate, lactose DT (direct tableting), lactose anhydrous, Flowlac™ (available from Meggle Products), Pharmatose™ (available from DMV) and others. Different grades of starches include but are not limited to maize starch, potato starch, rice starch, wheat starch, pregelatinized starch (commercially available as PCS PC10 from Signet Chemical Corporation) and Starch 1500, Starch 1500 LM grade (low moisture content grade) from Colorcon, fully pregelatinized starch (commercially available as National 78-1551 from Essex Grain Products) and others. Different cellulose compounds that can be used include crystalline celluloses and powdered celluloses. Examples of crystalline cellulose products include but are not limited to CEOLUS™ KG801, Avicel™ PH101, PH102, PH1 13, PH200, PH301, PH302 and PH-F20, PH1 12, microcrystalline cellulose 114, and microcrystalline cellulose 112. Other useful diluents include but are not limited to carmellose, sugar alcohols such as sorbitol and xylitol, calcium carbonate, magnesium carbonate, dibasic calcium phosphate, and tribasic calcium phosphate.

Suitable binders include one or more of povidones, starches, corn starch, pregelatinized starch, microcrystalline celluloses (MCC), silicified MCC (e.g., Prosolv™ HD 90), microfine celluloses, lactose, calcium carbonate, calcium sulfate, sugar, mannitol, sorbitol, dextrates, dextrin, maltodextrin, dextrose, dibasic calcium phosphate dihydrate, tribasic calcium phosphate, magnesium carbonate, magnesium oxide, stearic acid, gums, hydroxypropyl methylcelluloses or hypromelloses (e.g., Klucel™ EF, Methocel™ E5 premium), and the like.

Suitable antioxidants include one or more of ascorbic acid, sodium pyrosulphite, glutathion, sorbic acid, butylated hydroxytoluene (BHT), propyl gallate, butylated hydroxyanisole (BHA), vitamin E, vitamin E TPGS and derivatives thereof (e.g., the commercially available Speziol® TPGS Pharma supplied by Cognis GmbH.), citric acid, and the like.

Suitable disintegrants include one or more of carmellose calcium (Gotoku Yakuhin Co., Ltd.), carboxymethylstarch sodium (Matsutani Kagaku Co., Ltd., Kimura Sangyo Co., Ltd., etc.), croscarmellose sodium (Ac-di-sol™) (FMC-Asahi Chemical Industry Co., Ltd.), crospovidones, examples of commercially available crospovidone products including but not limited to crosslinked povidones, Kollidon™ CL [manufactured by BASF (Germany)], Polyplasdone™ XL, XI-10, and INF-10 [manufactured by ISP Inc. (USA)], and low-substituted hydroxypropylcelluloses. Examples of low-substituted hydroxypropylcelluloses include but are not limited to low-substituted hydroxypropylcellulose LH11, LH21, LH31, LH22, LH32, LH20, LH30, LH32 and LH33 (all manufactured by Shin-Etsu Chemical Co., Ltd.). Other useful disintegrants include sodium starch glycolate, starches, and the like.

Suitable surfactants include one or more of anionic, non-ionic or cationic surfactants. Useful surfactants are exemplified by, but not limited to, one or more of polyoxyethylene hardened castor oil, glycerin monostearate, sorbitan monostearate, sorbitan monopalmitate, sorbitan monolaurate, polyoxyethylene-polyoxypropylene block copolymers, polysorbates, sodium lauryl sulfate, macrogols, sucrose fatty acid esters, and the like.

Suitable lubricants include one or more of magnesium stearate, zinc stearate, calcium stearate, stearic acid, sodium stearyl fumarate, hydrogenated castor oil, and the like.

Suitable glidants include one or more of colloidal silicon dioxide, talc, corn starch, and the like.

Suitable coloring agents can be used to color code the formulation, for example, to indicate the type and dosage of the therapeutic agent therein. Suitable coloring agents include, without limitation, natural and/or artificial compounds such as FD&C coloring agents, natural juice concentrates, pigments such as titanium oxide, silicon dioxide, iron oxides, zinc oxide, combinations thereof, and the like.

The pharmaceutical formulations of the present invention optionally have one or more coatings, which are functional or non-functional. Functional coatings include modified release coatings and non-functional coatings include seal coatings and elegant coatings. Additional excipients such as film forming polymers, solvents, plasticizers, antiadherents, opacifiers and optionally colorants, pigments, antifoam agents, polishing agents can be used in coatings.

Suitable film-forming agents include but are not limited to cellulose derivatives such as soluble alkyl- or hydroalkyl-cellulose derivatives such as methylcelluloses, hydroxymethyl celluloses, hydroxyethyl celluloses, hydroxypropyl celluloses, hydroxymethylethyl celluloses, hydroxypropyl methylcelluloses, sodium carboxymethyl celluloses, etc., insoluble cellulose derivatives such as ethylcelluloses and the like, dextrins, starches and starch derivatives, polymers based on carbohydrates and derivatives thereof, natural gums such as gum Arabic, xanthans, alginates, polyacrylic acids, polyvinyl alcohols, polyvinyl acetates, polyvinylpyrrolidones, polymethacrylates and derivatives thereof (Eudragit™ products), chitosan and derivatives thereof, shellac and derivatives thereof, waxes and fat substances. Useful enteric coating materials include but are not limited to materials such as cellulosic polymers like cellulose acetate phthalates, cellulose acetate trimellitates, hydroxypropyl methylcellulose phthalates, polyvinyl acetate phthalates, etc., methacrylic acid polymers and copolymers (Eudragit™), and the like, and mixtures thereof.

Some of the excipients are used as adjuvants to the coating process, including excipients such as plasticizers, opacifiers, antiadhesives, polishing agents, and the like.

Suitable plasticizers include but are not limited to castor oil, diacetylated monoglycerides, dibutyl sebacate, diethyl phthalate, glycerin, polyethylene glycols, propylene glycols, triacetin, triethyl citrate, and mixtures thereof. An opacifier like titanium dioxide may also be present in an amount ranging from about 10% (w/w) to about 20% (w/w) based on the total weight of the coating.

Antiadhesives are frequently used in the film coating process to avoid sticking effects during film formation and drying. An example of an antiadhesive for this purpose is talc.

Suitable polishing agents include polyethylene glycols of various molecular weights or mixtures thereof, talc, surfactants (e.g. glycerol monostearate and poloxamers), fatty alcohols (e.g., stearyl alcohol, cetyl alcohol, lauryl alcohol and myristyl alcohol) and waxes (e.g., carnauba wax, candelilla wax and white wax).

Suitable solvents can be used in the processes of preparing pharmaceutical formulations of the present invention, including but not limited to, water, methanol, ethanol, acidified ethanol, acetone, diacetone, polyols, polyethers, oils, esters, alkyl ketones, methylene chloride, isopropyl alcohol, butyl alcohol, methyl acetate, ethyl acetate, isopropyl acetate, castor oil, ethylene glycol monoethyl ether, diethylene glycol monobutyl ether, diethylene glycol monoethyl ether, dimethylsulphoxide, N,N-dimethylformamide, tetrahydrofuran and mixtures thereof.

In addition to the above coating ingredients, sometimes preformulated coating products such as those sold as OPADRY™, Opadry™ II and Opadry™® AMB (supplied by Colorcon) or TABCOAT™ can be used. OPADRY formulations generally comprise polymer, plasticizer and, if desired, pigment in a dry concentrate. OPADRY products produce attractive, elegant coatings on a variety of tablet cores and can be used in both aqueous and organic coating procedures. Preformulated coating products sold in solid form generally require only dispersion in a liquid before use.

The foregoing descriptions of excipients are not intended to be exhaustive. Those skilled in the art will be aware of many other substances that are useful in the practice of the invention, and the use of such substances is specifically included in this invention.

In embodiments, the invention includes formulations comprising clopidogrel or a pharmaceutically acceptable salt thereof as active agent, together with one or more pharmaceutically acceptable excipients, wherein the active agent is present in an amount of about 30-450 mg in a unit dose of the formulation.

In yet another aspect, the invention includes methods of preparing the formulations of the present invention.

In an embodiment, a method for preparing the pharmaceutical formulations comprises the steps of treating clopidogrel or pharmaceutically acceptable salts thereof together with one or more pharmaceutically acceptable excipients and preparing into a suitable dosage form.

Embodiments of the processes of preparing the pharmaceutical formulations comprise the step of spray drying a suspension or solution of clopidogrel or its salt and optionally one or more pharmaceutically acceptable excipients, to obtain a spray-dried product which is further processed to make a desired formulation.

Embodiments of the processes of preparing the pharmaceutical formulations comprise the use of a fluid bed coating technique, wherein a solution of clopidogrel or its salt, optionally together with one or more pharmaceutically acceptable excipients, is sprayed onto an inert core or layered on an inert core which is further processed to make the desired formulation.

Embodiments of the processes of preparing the pharmaceutical formulations comprise: (a) sifting diluents, disintegrants, and optionally one or more other excipients, and mixing; (b) preparing a granulating solution by dispersing or dissolving active agent in a solvent; (c) granulating the material of step (a) with granulating solution of step (b) and drying the granules; (d) sifting the dried granules and desired extragranular excipients through a sieve; (e) blending sifted granules and extragranular excipients and adding a lubricant to the blend; (f) compressing the lubricated blend into tablets; and (g) optionally coating the tablets of step (f).

Embodiments of processes may include formulation preparation using direct compression, comprising forming a blend by mixing the clopidogrel or a pharmaceutically acceptable salt thereof with a diluent, a disintegrant, and optionally one or more conventional excipients. The above blend can be further processed into the desired oral dosage forms such as tablets, capsules or pellets.

Embodiments of direct compression include slugging or roller compaction. Slugging is a double compression process. The material to be tableted is compressed into a large compressed mass, or “slug,” which is broken up and converted to tablets by a second compression process. Roller compaction has the benefits of a granulation process, such as improved material flow behavior and content uniformity. In addition, roller compaction is high-volume and can be more economical to operate than wet granulation.

Alternatively, wet granulation may be used to obtain granules with good handling properties. The granules may then be further processed into a desired form, such as a tablet.

In an embodiment, the invention includes stable pharmaceutical formulations of clopidogrel or its salts, prepared comprising the step of spray drying a suspension or solution of clopidogrel or its salt and optionally one or more pharmaceutically acceptable excipients to obtain a spray-dried product, which is further processed to make the desired formulation. Alternatively, clopidogrel or its salt containing formulations may also be prepared by a fluid bed granulation technique, wherein a solution of clopidogrel or its salt, optionally together with one or more pharmaceutically acceptable excipients, is sprayed onto pharmacologically inert excipient blends.

Dosage forms prepared as above can be subjected to in vitro dissolution evaluations according to Test 711 “Dissolution” in United States Pharmacopoeia 29, United States Pharmacopeial Convention, Inc., Rockville, Md., 2005 (“USP”), to determine the rate at which the active substance is released from the dosage forms. The content of active substance and the impurity levels are conveniently measured by techniques such as high performance liquid chromatography (HPLC).

In some embodiments, the invention includes use of packaging materials such as containers and closures of high-density polyethylene (HDPE), low-density polyethylene (LDPE) and or polypropylene and/or glass, glassine foil, aluminum pouches, and blisters or strips composed of aluminum or high-density polyvinyl chloride (PVC), optionally including a desiccant, polyethylene (PE), polyvinylidene dichloride (PVDC), PVC/PE/PVDC, and the like.

The pharmaceutical dosage forms of the present invention are intended for oral administration to a patient in need thereof.

In a further aspect the invention includes methods of treating patients suffering from atherothrombotic events (e.g., myocardial infarction, stroke and vascular death) or acute coronary syndrome using the pharmaceutical formulations of the present invention.

Pharmaceutical formulations of the present invention can optionally be administered with one, or more than one, other therapeutic agents in the treatment of thrombotic disorders, including, but not limited to: salicylates such as aspirin; angiotensin II receptor antagonists such as candesartan, valsartan, eprosartan, losartan, irbesartan, saprisartan, zolasartan, saralasin, telmisartan, tasosartan; isoteoline; HMG CoA reductase inhibitors such as atorvastatin, cerivastatin, fluvastatin, lovastatin, pravastatin, rosuvastatin, simvastatin, pitvastatin, fluindostatin, mevastatin, velostatin and dalvastatin; folic acid; vitamins; and their pharmaceutically acceptable salts, solvates, hydrates, enantiomers, etc. Pharmaceutical formulations of the present invention also can optionally be administered with antidiabetes agents, including sulfonylureas, meglitinide, nateglinide, a biguanide, a thiazolidinedione, or an alpha-glucose inhibitor. The different agents that can be used are well known to those skilled in the art, and the use of any of them falls within the scope of the invention.

Certain specific aspects and embodiments of the invention will be described in more detail with reference to the following examples, being provided only for purposes of illustration, and it is to be understood that the present invention is not deemed to be limited thereto.

### EXAMPLE 1

### Clopidogrel Premix Composition

| Ingredient | Grams |
|---|---|
| Clopidogrel base | 75 |
| Butylated hydroxyanisole (BHA) | 1.2 |
| Mannitol (Pearlitol ™ SD 200) | 25 |
| Lactose anhydrous (Pharmatose ™ DCL | 175 |
| 21) | |
| Microcrystalline cellulose (Avicel ™ PH | 90 |
| 112) | |
| Colloidal silicon dioxide (Aerosil ™ 200) | 20 |
| Isopropyl alcohol* | 172.5 |

*Evaporates during processing.

Manufacturing Process:

1. Dissolve clopidogrel base and BHA in isopropanol with stirring.

2. Sift mannitol, lactose anhydrous, microcrystalline cellulose and colloidal silicon dioxide through an ASTM 40 mesh sieve and mix for 10 to 15 minutes.

3. Mix the solution of step 1 with the blend of step 2 thoroughly.

4. Dry the wet mass of step 3 at 40-45° C. under 600 mm Hg vacuum for 2 to 3 hours, until a loss on drying at 105° C. of 1-2 percent by weight is achieved.

5. Sift dried granules through an ASTM 24 mesh sieve to obtain the premix.

### EXAMPLE 2

### Clopidogrel Premix Composition

| Ingredient | Grams |
|---|---|
| Clopidogrel base | 75 |
| Butylated hydroxyanisole (BHA) | 1.2 |
| Mannitol (Pearlitol SD 200) | 25 |
| Lactose anhydrous (Pharmatose DCL 21) | 87.5 |
| Sucrose | 87.5 |
| Microcrystalline cellulose (Avicel PH 112) | 90 |
| Colloidal silicon dioxide (Aerosil 200) | 20 |
| Isopropyl alcohol* | 172.5 |

*Evaporates during processing.

Manufacturing Process:

1. Dissolve clopidogrel base and BHA in isopropanol with stirring.

2. Sift mannitol, lactose anhydrous, microcrystalline cellulose and colloidal silicon dioxide and sucrose through an ASTM 40 mesh sieve and mix for 10 to 15 minutes.

3. Mix the solution of step 1 with the blend of step 2 thoroughly.

4. Evaporate solvent using a Buchi Rotavapor at a temperature of 37° C. and a pressure of 36 mm Hg, and then sift through a sieve to obtain the premix.

### EXAMPLE 3

### Clopidogrel 75 mg Tablets Containing Sodium Stearyl Fumarate as a Lubricant

| Ingredient | mg/Tablet |
|---|---|
| Clopidogrel premix (Example 1) | 386.2* |
| Silicon dioxide (Syloid ® 244 FP) | 13 |
| Microcrystalline cellulose (Avicel PH 200) | 182.44 |
| Mannitol (Pearlitol SD 200) | 57.61 |
| Croscarmellose sodium | 1 |
| Sodium stearyl fumarate | 9.75 |

Film Coating

| | |
|---|---|
| Opadry ™ II Pink 85G4039** | 26 |
| Water‡ | 182 |

*Based on the assay of clopidogrel in the premix batch.
**OPADRY II Pink 85G4039 is a preformulated coating product containing polyvinyl alcohol, talc, titanium dioxide, macrogol/PEG 3350, lecithin (soya) and iron oxide red, supplied by Colorcon.
‡Evaporates during processing.

Manufacturing Process:

1. Sift clopidogrel premix through an ASTM 20 mesh sieve and sift silicon dioxide through an ASTM 60 mesh sieve.

2. Blend the sifted materials of step 1 in a double cone blender for 5 minutes.

3. Sift microcrystalline cellulose and mannitol through an ASTM 30 mesh sieve, add to the material of step 2, and blend for 5 minutes.

4. Sift croscarmellose sodium through an ASTM 30 mesh sieve, sift sodium stearyl fumarate through an ASTM 60 mesh sieve, and blend with the step 3 mixture for 10 minutes.

5. Compress the blend of step 4 into tablets.

6. Coat the tablets of step 5 using Opadry® II Pink 85G 54039 coating dispersion to produce a weight gain of about 3-5% w/w after drying.

The formulation is subjected to stability testing, in which tablets are filled into high-density polyethylene (HDPE) bottles with caps and stored at 40° C. and 75% relative humidity (RH) conditions for 6 months, and are analyzed at intervals to evaluate the extent of impurity formation. Values in the table below are expressed as percentages of the label drug content.

| | Percent by Weight | | | |
|---|---|---|---|---|
| Impurity | Initial | 1 Month | 3 Months | 6 Months |
| Impurity A | 0.007 | 0.008 | 0.01 | 0.02 |
| Impurity B | 0.05 | 0.07 | 0.1 | 0.15 |
| Impurity C | BLD | 0.01 | 0.03 | BLD |
| Impurity D | 0.008 | 0.06 | 0.03 | 0.02 |
| Highest | 0.12 | 0.04 | 0.05 | 0.11 |
| unidentified | | | | |
| impurity | | | | |
| Total impurities | 0.09 | 0.36 | 0.56 | 0.82 |

BLD = Below the limit of detection.

Impurity A has a chemical name (+)-(S)-(o-chlorophenyl)-6,7-dihydrothieno-[3,2-c]pyridine-5 (4H)-acetic acid, and the structure below.

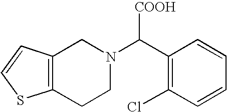

Impurity B has a chemical name Thieno [3,2-c]-4,5,6,7-tetrahydropyridine hydrochloride, and the structure below.

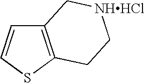

Impurity C has a chemical name 2-chloro-a-[2-(2-thiene)ethyl]amino]benzene acetic acid methyl ester bisulfate, and the structure below.

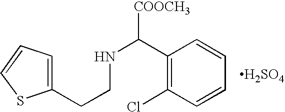

Impurity D has a chemical name 6,7-dihydrothieno[3,2-c] pyridine-4(5H)-one, and the structure below.

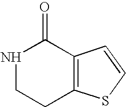

According to the International Conference on Harmonization (ICH) guidelines for low dose drugs, the individual impurities should not exceed 0.5% of the label drug content during the course of stability testing. The levels of impurities with this formulation are within these acceptable levels.

### EXAMPLE 4

### Clopidogrel 75 mg Tablets Containing Magnesium Stearate as a Lubricant

| Ingredient | mg/Tablet |
|---|---|
| Clopidogrel premix (Example 2) | 382.65* |
| Microcrystalline cellulose | 159.23 |
| Mannitol | 49.12 |
| Colloidal silicon dioxide | 3 |
| Magnesium stearate | 6 |

*Based on the assay of clopidogrel in the premix batch.

Manufacturing Process:

1. Blend the microcrystalline cellulose, mannitol and colloidal silicon dioxide thoroughly.

2. Add clopidogrel premix to the material of step 1 and blend.

3. Add magnesium stearate to the material of step 2 and blend.

4. Compress the blend of step 3 into tablets.

### EXAMPLE 5

### Clopidogrel 75 mg Tablets Containing Hydrogenated Castor Oil as a Lubricant

| Ingredient | mg/Tablet |
|---|---|
| Clopidogrel premix (Example 1) | 363* |
| Microcrystalline cellulose | 159.75 |
| Mannitol | 53.25 |
| Colloidal silicon dioxide | 12 |
| Croscarmellose sodium | 12 |
| Hydrogenated castor oil | 12 |

*Based on the assay of clopidogrel in the premix batch.

Manufacturing Process:

1. Blend the microcrystalline cellulose, mannitol, colloidal silicon dioxide and croscarmellose sodium thoroughly.

2. Add clopidogrel premix to the material of step 1 and blend.

3. Add hydrogenated castor oil to the material of step 2 and blend.

4. Compress the blend of step 3 into tablets.

Tablets are filled into closed high-density polyethylene (HDPE) bottles and stored under 40° C. and 75% relative humidity (RH) conditions for 3 months, and are analyzed to evaluate the extent of impurity formation. Also, tablets are stored unpackaged under 40° C./75% RH and 50° C. conditions for 4 weeks and analyzed to determine the degree of impurity formation during storage. Values in the table below are expressed as percentages of the label drug content.

| | Percent by Weight | | | |
|---|---|---|---|---|
| | | 40° C./75% RH* | 50° C.* | 40° C./75% RH** |
| Impurity | Initial | 4 Weeks | 4 Weeks | 3 Months |
| Impurity A | BLD | BLD | BLD | BLD |
| Impurity B | 0.03 | 0.07 | 0.12 | 0.09 |
| Impurity C | BLD | 0.01 | 0.01 | 0:01 |
| Impurity D | 0.01 | BLD | BLD | 0.02 |
| Highest | 0.03 | 0.04 | 0.16 | 0.12 |
| unidentified | | | | |
| impurity | | | | |
| Total | 0.07 | 0.25 | 0.67 | 0.45 |
| impurities | | | | |

*Unpackaged.
**Packaged in HDPE bottles.
BLD = Below the limit of detection.

### EXAMPLE 6

### Clopidogrel 75 mg Tablets Containing Sodium Stearyl Fumarate as a Lubricant

mg/Tablet

| Ingredient | 6A | 6B |
|---|---|---|
| Clopidogrel premix (Example 1) | 382.65* | 382.65* |
| Microcrystalline cellulose | 162.11 | 159.17 |
| Mannitol | 49.12 | 49.12 |
| Colloidal silicon dioxide | 3.06 | 3.06 |
| Sodium stearyl fumarate | 3.06 | 6 |

*Based on the assay of clopidogrel in the premix batch.

Manufacturing Process:

1. Blend microcrystalline cellulose, mannitol and colloidal silicon dioxide thoroughly.

2. Add clopidogrel premix to the material of step 1 and blend.

3. Add sodium stearyl fumarate to the material of step 2 and blend.

4. Compress the blend of step 3 into tablets.

Tablets are stored unpackaged under 40° C. and 75% RH, and 50° C., conditions for 4 weeks and analyzed to determine the degree of impurity formation during storage. Values in the table below are expressed as percentages of the label drug content.

| | Percent by Weight | | | | | |
|---|---|---|---|---|---|---|
| | 6A | | | 6B | | |
| | | 40° C./ | | | 40° C./ | |
| Impurity | Initial | 75% RH | 50° C. | Initial | 75% RH | 50° C. |
| Impurity A | BLD | 0.003 | 0.004 | BLD | BLD | BLD |
| Impurity B | 0.05 | 0.07 | 0.01 | 0.08 | 0.15 | 0.19 |
| Impurity C | BLD | 0.01 | BLD | BLD | BLD | BLD |
| Impurity D | 0.01 | BLD | BLD | BLD | BLD | BLD |
| Highest | 0.10 | 0.05 | 0.26 | 0.09 | 0.09 | 0.24 |
| unidentified | | | | | | |
| impurity | | | | | | |
| Total | 0.30 | 0.25 | 0.75 | 0.29 | 0.44 | 0.85 |
| impurities | | | | | | |

BLD = Below the limit of detection.

### EXAMPLE 7

### Clopidogrel 300 mg Formulation

| | |
|---|---|
| Ingredient | mg/Tablet |

Dry Blend

| | |
|---|---|
| Clopidogrel bisulphate | 391.5 |
| Microcrystalline cellulose (Avicel PH112) | 438.5 |
| Mannitol (Perlitol SD 200) | 75 |
| Croscarmellose sodium | 15 |
| Hydroxypropyl cellulose (Klucel EF) | 40 |
| Hydroxypropyl methylcellulose (HPMC 5 cps) | 20 |

Lubrication

| | |
|---|---|
| Hydrogenated castor oil powder | 20 |

Final Coating

| | |
|---|---|
| Opadry Pink 03B54202# | 37.65 |
| Polyethylene glycol 400 (PEG 400) | 2.35 |
| Water* | q.s. |

#Opadry Pink 03B54202 is a pre-formulated coating product containing HPMC 2910/Hypromellose, titanium dioxide, PEG 400 and iron oxide red, supplied by Colorcon.
*Evaporates during processing.

Manufacturing Process:

1. Sift Klucel EF through an ASTM 20 mesh sieve.

2. Sift together clopidogrel bisulphate, microcrystalline cellulose, mannitol, croscarmellose and hydroxypropyl methylcellulose through an ASTM 40 mesh sieve.

3. Sift hydrogenated castor oil powder through an ASTM 60 mesh sieve.

4. Mix the materials of steps 1 and 2 in a double cone blender for 5 minutes.

5. Add the sifted hydrogenated castor oil to material of step 4 and further blend for 5 minutes.

6. Compress the blend from step 5 into tablets.

7. Dissolve the polyethylene glycol 400 in water and disperse Opadry Pink in it using a mechanical stirrer, and continue the stirring for 45 minutes.

8. Coat the tablets of step 6, using coating suspension of step 7 until the weight build-up is about 2.5±0.5% w/w after drying.

Two in vivo pharmacokinetic studies with the prepared tablets (“T”), and the marketed PLAVIX® clopidogrel 300 mg product (“R”), are conducted with healthy human subjects and the results are presented below.

Number of subjects: 67; type of study: single dose, fed-state, 2-way crossover.

| Parameter | Mean T ÷ R × 100 | 90% Confidence Interval |
|---|---|---|
| AUC₀₋ₜ | 98.7 | 94.64-102.98 |
| AUC_{0-∞} | 98.7 | 94.61-102.92 |
| Cₘₐₓ | 97.8 | 91.18-104.87 |

Number of subjects: 66; type of study: single dose, fasted state, 2-way crossover.

| Parameter | Mean T ÷ R × 100 | 90% Confidence Interval |
|---|---|---|
| AUC₀₋ₜ | 93.1 | 87.23-102.33 |
| AUC_{0-∞} | 94.5 | 85.99-100.89 |
| Cₘₐₓ | 89.4 | 81.25-98.48 |

Prepared tablets are filled into high-density polyethylene (HDPE) bottles, closed with caps and stored under 40° C. and 75% relative humidity (RH) conditions for 6 months, and are analyzed at intervals to evaluate the extent of impurity formation and moisture content.

| | Percent by Weight | | | | |
|---|---|---|---|---|---|
| Impurity | Initial | 1 Month | 2 Months | 3 Months | 6 Months |
| Impurity A | 0.06 | 0.08 | 0.08 | 0.11 | 0.16 |
| Impurity B | BLD | BLD | 0.06 | BLD | BLD |
| Impurity C | BLD | 0.03 | 0.03 | 0.02 | 0.02 |
| Impurity D | 0.02 | 0.02 | 0.02 | 0.02 | 0.03 |
| Highest | 0.03 | 0.03 | 0.03 | 0.03 | 0.03 |
| unidentified | | | | | |
| impurity | | | | | |
| Total impurities | 0.18 | 0.22 | 0.27 | 0.29 | 0.31 |
| Water content | 3.0 | 2.4 | 2.5 | 2.6 | 2.3 |
| by KF | | | | | |

BLD = Below the limit of detection.

Prepared tablets are filled into blister packages (aluminum foil, paper backed, peeleable) and stored under 40° C. and 75% RH conditions for 6 months, and are analyzed to evaluate the extent of impurity formation and moisture content.

Percent by Weight

| Impurity | Initial | 6 Months |
|---|---|---|
| Impurity A | 0.06 | 0.17 |
| Impurity B | BLD | BLD |
| Impurity C | BLD | BLD |
| Impurity D | 0.02 | 0.03 |
| Highest unidentified impurity | 0.03 | 0.05 |
| Total impurities | 0.18 | 0.38 |
| Water content by KF | 3.0 | 2.3 |

BLD = Below the limit of detection.

Prepared tablets are packaged in closed HDPE containers and stored under accelerated stability testing conditions of 40° C. and 75% RH for 3 months, and the formulations are analyzed for polymorphic stability by XRD. The clopidogrel bisulphate is found to be in polymorphic Form I, both before and after the storage.

### EXAMPLE 8

### Clopidogrel 300 mg Formulation

| | |
|---|---|
| Ingredient | mg/Tablet |

Dry Blend

| | |
|---|---|
| Clopidogrel bisulphate | 391.5 |
| Microcrystalline cellulose (Avicel ® PH112) | 438.5 |
| Mannitol (Perlitol ® SD 200) | 75 |
| Croscarmellose sodium | 15 |
| Hydroxypropyl cellulose (Klucel ® EF) | 40 |
| Hydroxypropyl methylcellulose (HPMC 5 cps) | 20 |

Lubrication

| | |
|---|---|
| Hydrogenated castor oil powder | 20 |

Film Coating

| | |
|---|---|
| HPMC 5 cps | 8 |
| Water* | q.s. |

Final Coating

| | |
|---|---|
| Opadry ® Pink 03B54202 | 28.24 |
| Polyethylene glycol 400 | 1.76 |
| Water* | q.s. |

*Evaporates during processing.

Manufacturing process: similar to that described in Example 7, except for an additional step of film coating using HPMC in water, prior to the final coating step.

We claim:

1. A pharmaceutical formulation comprising clopidogrel or a salt thereof as an active agent, and about 0.5 to about 5 percent by weight of a lubricant comprising one or more of stearic acid, magnesium stearate, zinc stearate, calcium stearate, sodium stearyl fumarate, glycery behenate and hydrogenated castor oil.

2. The pharmaceutical formulation of claim 1, further comprising a diluent comprising microcrystalline cellulose, a disintegrant comprising one or more of croscarmellose sodium, sodium starch glycolate and crospovidone, and a lubricant comprising one or both of sodium stearyl fumarate and hydrogenated castor oil.

3. The pharmaceutical formulation of claim 1, wherein a lubricant comprises sodium stearyl fumarate.

4. The pharmaceutical formulation of claim 1, having a water content about 0.5 to 10 percent by weight, as determined using the Karl Fischer method.

5. The pharmaceutical formulation of claim 1, having a loss on drying at 40-250° C. less than about 12 percent by weight.

6. The pharmaceutical formulation of claim 1, wherein the active agent has a particle size distribution with D90 about 0.1 μto about 1000 μ, and D50 about 0.01 μm to about 500 μ.

7. The pharmaceutical formulation of claim 1, wherein total impurities are less than about 5 percent of the label clopidogrel content, after storage at 40° C. and 75% relative humidity for 2 months.

8. The pharmaceutical formulation of claim 1, wherein the content of an impurity having the structure 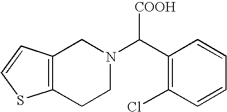
is less than about 1 percent of the label clopidogrel content, after storage at 40° C. and 75% relative humidity for 2 months.

9. The pharmaceutical formulation of claim 1, wherein an active agent comprises clopidogrel base.

10. The pharmaceutical formulation of claim 1, wherein an active agent comprises a solid premix comprising clopidogrel base, adsorbed onto at least one pharmaceutically acceptable excipient.

11. The pharmaceutical formulation of claim 1, wherein an active agent comprises clopidogrel bisulphate.

12. The pharmaceutical formulation of claim 1, wherein an active agent comprises clopidogrel bisulphate Form I.

13. The pharmaceutical formulation of claim 1, being substantially free of clopidogrel bisulphate Form II.

14. The pharmaceutical formulation of claim 1, further comprising, as an active agent, at least one of an antihypertensive agent, a salicylate, a HMG CoA reductase inhibitor, an antidiabetes agent, folic acid, and a vitamin.

15. A method of treating an atherothrombotic event or acute coronary syndrome, comprising administering a pharmaceutical formulation of claim 1.

16. The method according to claim 15, wherein an atherothrombotic event includes one or more of myocardial infarction, stroke and vascular death.

17. A pharmaceutical tablet comprising clopidogrel base, adsorbed onto at least one pharmaceutically acceptable excipient, as an active agent, and about 0.5 to about 5 percent by weight of a lubricant comprising sodium stearyl fumarate.

18. The pharmaceutical tablet of claim 17, wherein total impurities are less than about 5 percent of the label clopidogrel content, after storage at 40° C. and 75% relative humidity for 2 months.

19. A pharmaceutical tablet comprising clopidogrel bisulphate as an active agent, and about 0.5 to about 5 percent by weight of a lubricant comprising sodium stearyl fumarate.

20. The pharmaceutical tablet of claim 19, wherein total impurities are less than about 5 percent of the label clopidogrel content, after storage at 40° C. and 75% relative humidity for 2 months.
